SEQUENCE LISTING (7 sequences)

SEQ ID NO: 1 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 160 aa, Artificial
  mouse IL-10 (mIL-10)
SRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 3 - protein, 145 aa, Artificial
  viral form of IL-10 (vIL-10)
QCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 4 - protein, 9 aa, artificial sequence
  synthetic amino acid sequence  IL-10-derived peptide
AYMTMKIRN

SEQ ID NO: 5 - protein, 9 aa, artificial sequence
  synthetic amino acid sequence IL-10-derived peptide
XXXTXKXRX

SEQ ID NO: 6 - protein, 13 aa, artificial sequence
  synthetic amino acid sequence C-terminal 13-mer
WITFCQSIISTLT

SEQ ID NO: 7 - protein, 9 aa, artificial sequence
  synthetic amino acid sequence “scrambled” peptide
RIKNMATYM

(19) United States
(12) Patent Application Publication
(10) Pub. No.: US 2010/0028296 A1
(43) Pub. Date: Feb. 4, 2010

(54) Use of cytokine-derived peptides in treatment of pain and neurodegenerative disease

(76) Inventors: Raymond A. Chavez, Alameda, CA (US); Kirk W. Johnson, Moraga, CA (US); Jennifer Shumilla, San Mateo, CA (US); Laura Sanftner, El Sobrante, CA (US)

Correspondence Address: ROBINS & PASTERNAK, 1731 EMBARCADERO ROAD, SUITE 230, PALO ALTO, CA, 94303, US

(21) Appl. No.: 11/919,108

(22) PCT Filed: May 1, 2006
(86) PCT No.: PCT/US2006/016594

§ 371 Date: Sep. 22, 2009

Related U.S. Application Data
(60) Provisional application No. 60/677,254, filed on May 2, 2005.
(60) Provisional application No. 60/720,567, filed on Sep. 26, 2005.

(51) Int. Cl. A61K 38/19; A61K 38/20; A61K 38/18; A61P 25/00

(52) U.S. Cl. 424/85.2; 514/2; 514/12

(57) ABSTRACT

Peptides derived from anti-inflammatory cytokines, such as IL-10, are disclosed for use in treatment of neurodegenerative diseases and neuropathic pain indications, including Alzheimer's disease, Amyotrophic Lateral Sclerosis, Multiple Sclerosis, Parkinson's disease and trigeminal neuralgia.

### CROSS-REFERENCE TO RELATED APPLICATION

This application claims the benefit under 35 USC§119(e)(1) of U.S. application Ser. Nos. 60/677,254, filed May 2, 2005, and 60/720,567, filed Sep. 26, 2005, which applications are incorporated herein by reference in their entireties.

### TECHNICAL FIELD

The present invention relates generally to therapeutic treatment of chronic neurological syndromes in humans. Specifically, the invention relates to treatment of pain, such as trigeminal neuralgia, and neurodegenerative diseases, using anti-inflammatory molecules such as IL-10-derived peptides, that act on proinflammatory cytokines.

### BACKGROUND

Activated spinal cord microglia and astrocytes appear to contribute to the creation and maintenance of pathological pain. In particular, activated glia appear to do so, at least in part, via their release of the proinflammatory cytokines interleukin-1β (IL-1β), tumor necrosis factor alpha (TNFα), and IL-6 (for review, see Watkins et al., Trends in Neurosci. (2001) 24:450-455). These proinflammatory cytokines amplify pain by enhancing the release of “pain” neurotransmitters from incoming sensory nerve terminals and by enhancing the excitability of spinal cord dorsal horn pain transmission neurons (Reeve et al., Eur. J. Pain (2000) 4:247-257; Watkins et al., Trends in Neurosci. (2001) 24:450-455).

Astrocytes and microglia express receptors for IL-10 (Mizuno et al., Biochem. Biophys. Res. Commun. (1994) 205:1907-1915) while spinal cord neurons do not (Ledeboer et al., J. Neuroimmunol. (2003) 136:94-103). In vitro studies have shown that IL-10 can selectively suppress proinflammatory cytokine production and signaling in these glial cells (Moore et al., Ann. Rev. Immunol. (2001) 19:683-765). In fact, IL-10 is an especially powerful member of the anti-inflammatory cytokine family in that it can suppress all proinflammatory cytokines implicated in pathological pain (IL-1β, TNFα and IL-6). IL-10 exerts this effect by inhibiting p38 MAP kinase activation (Strle et al., Crit. Rev. Immunol. (2001) 21:427-449); inhibiting NFkappaB activation, translocation and DNA binding (Strie et al., Crit. Rev. Immunol. (2001) 21:427-449); inhibiting proinflammatory cytokine transcription (Donnelly et al., J. Interferon Cytokine Res. (1999) 19:563-573; inhibiting proinflammatory cytokine mRNA stability and translation (Hamilton et al., Pathobiology (1999) 67:241-244; Kontoyiannis et al., EMBO J (2001) 20:3760-3770); and inhibiting proinflammatory cytokine release (Moore et al., Ann. Rev. Immunol. (2001) 19:683-765). In addition, IL-10 stabilizes mRNAs of suppressors of cytokine signaling, thereby increasing the production of a family of proteins that further inhibit proinflammatory cytokine production (Strie et al., Crit. Rev. Immunol. (2001) 21:427-449). IL-10 also interrupts proinflammatory cytokine signaling by downregulating proinflammatory cytokine receptor expression (Sawada et al., J. Neurochem. (1999) 72:1466-1471). Lastly, it upregulates endogenous antagonists of proinflammatory cytokines, including IL-1β receptor antagonist and TNFα decoy receptors (Foey et al., J Immunol. (1998) 160:920-928; Huber et al., Shock (2000) 13:425-434).

The known effects of IL-10 are restricted to suppression of proinflammatory functions of activated immune and glial cells, leaving non-inflammatory aspects of cellular functions unaffected (Moore et al., Ann. Rev. Immunol. (2001) 19:683-765). While some neurons express IL-10 receptors, the only known action of IL-10 on neurons is inhibition of cell death (apoptosis) (Bachis et al., J. Neurosci. (2001) 21:3104-3112).

Laughlin et al. (Laughlin et al., Pain (2000) 84:159-167) reported that intrathecal IL-10 blocks the onset of intrathecal dynorphin-induced, IL-1β-mediated mechanical allodynia when administered 30 minutes or 6 hours after induction of dynorphin-induced allodynia, but not when administered 24 hours after induction. In the clinical setting it is preferable that any treatment for neuropathic pain be capable of reversing pre-existing allodynia, rather than merely preventing the establishment of allodynia, since patients presenting with allodynia symptoms will, by definition, have existing allodynia. These investigators then tested the effect of IL-10 on pathological pain induced by excitotoxic spinal cord injury, a manipulation that activates astrocytes and microglia at the site of injury (Brewer et al., Exp. Neurol. (1999) 159:484-493). IL-10 decreased pathological pain behaviors when given 30 minutes following injury (Plunkett et al., Exper. Neurol. (2001) 168:144-154; Yu et al., J. Pain (2003) 4:129-140). This is in keeping with the fact that systemic IL-10 can reduce spinal cord proinflammatory cytokine production in response to excitotoxic injury, a manipulation that allows systemic IL-10 to reach the injured spinal cord due to disruption of the blood-brain barrier (Crisi et al., Eur. J. Immunol. (1995) 2:3033-3040; Bethea et al., Neurotrauma (1999) 16:851-863).

One example of chronic pain is trigeminal neuralgia (TGN, also known as tic douloureux), a disorder of the fifth cranial (trigeminal) nerve that causes episodes of intense, stabbing, electric shock-like pain in the areas of the face where the branches of the nerve are distributed—lips, eyes, nose, scalp, forehead, upper jaw, and lower jaw. TGN has an estimated prevalence of 155 cases per million persons. A less common form of the disorder called Atypical Trigeminal Neuralgia may cause less intense, constant, dull burning or aching pain, sometimes with occasional electric shock-like stabs. Both forms of the disorder most often affect one side of the face, but some patients experience pain at different times on both sides. Onset of symptoms occurs most often after age 50, but cases are known in children and even infants. Something as simple and routine as brushing the teeth, putting on makeup or even a slight breeze can trigger an attack, resulting in agony for the individual. TGN is not fatal, but it is universally considered to be the most painful affliction known to medical practice. Initial treatment of TGN is usually by means of anti-convulsant drugs, such as carbamazepine (Tegretol®) or gabapentin (Neurontin®). Some anti-depressant drugs also have significant pain relieving effects. Should medication be ineffective or if it produces undesirable side effects, invasive neurosurgical procedures can be used to relieve pressure on the nerve or to reduce nerve sensitivity.

IL-10 may also be of use in the treatment of neurodegenerative disorders, such as Alzheimer's disease (AD), Parkinson's disease (PD), amyotrophic lateral sclerosis (ALS) and multiple sclerosis (MS), which will become increasingly prevalent in the United States as the population ages. Recent research has found a common thread in these conditions, viz. neuroinflammation due to glial activation, that has been extended to other neurodegenerative conditions (Griffin WST, et al. (1989) Proc Natl Acad Sci USA 86:7611-7615; Rogers J, et al. (1988) Neurobiol Aging 9:339-349; Akiyama H, et al. (2000) Neurobiol Aging 21:383-421; Eikelenboom P, et al. (2002) Glia 40:232-239; Orr C F, et al. (2002) Progr Neurobiol 68:325-340; Ishizawa K, and Dickson D W (2001) J Neuropathol Exp Neurol 60:647-657). Further discussion of the use of IL-10 for treatment of neurodegenerative disorders is found in U.S. Pat. No. Publication No. 2006/0073119, the disclosure of which is hereby incorporated by reference in its entirety.

Despite the potential beneficial effects in central nervous system (CNS) disorders, delivery of IL-10 systemically to treat CNS disorders is problematic. Administration of full-length IL-10 protein is the most direct form of therapy. Currently, commercially available IL-10 protein is synthesized in cultured bacterial, yeast, insect, mammalian, or other cells and delivered to patients by intravenous injection. Intravenous injection of recombinant proteins has been successful but suffers from several drawbacks. First, because IL-10 has a short half life, sustained delivery for prolonged periods would be difficult (Radwanski et al., Pharm. Res. (1998) 15:1895-1901). Patients would require several injections each single day to maintain the necessary levels of the protein in the blood stream. Even then, the concentration of protein would not be maintained at steady physiological levels (e.g. the level of the protein may be abnormally high immediately following an injection and far below optimal levels immediately prior to a subsequent injection).

Second, delivery to the appropriate target cells, tissue or organ can be problematic. IL-10 has not been successfully delivered orally, and thus presents problems for systemic administration. Intravenous delivery may fail to effect delivery to the desired target cell or tissue, particularly the brain, where the blood-brain-barrier effectively precludes delivery of therapeutic levels of all but the smallest protein molecules. IL-10, which exists as a dimer (approximately 37 kDa) in its native state, does not cross the intact blood brain barrier in appreciable amounts (Banks, W. A., J. Neurovirol. (1999) 5:538-555). Intravenous injection, even if successful, is inconvenient and can severely restrict a patient's lifestyle in cases where chronic treatment is required.

Gene therapy has also been used to deliver IL-10. IL-10 gene therapy reduces pneumonia-induced lung injury (Morrison et al., Infect. Immun. (2000) 68:4752-4758), decreases the severity of rheumatoid arthritis (Ghivizzani et al., Clin. Orthop. (2000) 379 Suppl.:S288-299), decreases inflammatory lung fibrosis (Boehler et al., Hum. Gene Ther. (1998) 9:541-551), inhibits cardiac allograft rejection (Brauner et al., J. Thoracic Cardiovasc. Surg. (1997) 114:923-933), suppresses endotoxemia (Xing et al., Gene Ther. (1997) 4:140-149), prevents and treats colitis (Lindsay et al., J. Immunol. (2001) 166:7625-7633), and reduces contact hypersensitivity (Meng et al., J. Clin. Invest. (1998) 101: 1462-1467). Use of IL-10 gene therapy to reverse ongoing pain is disclosed in WO 2005/000215, the disclosure of which is hereby incorporated by reference in its entirety.

Even if successfully delivered systemically, IL-10 would also disrupt the normal functions of the body's immune system and would be expected to be detrimental to the health of the patient (Xing et al., Gene Ther. (1997) 4:140-149; Fedorak et al., Gastroenterol. (2000) 119:1473-1482; Tilg et al., J. Immunol. (2002) 169:2204-2209).

The need exists for compositions and methods for providing IL-10 activity to the CNS of subjects in need of immunomodulatory therapy, such as those suffering from neuropathic pain (e.g. TGN) and neurodegenerative disease (e.g. AD, PD, ALS and MS). The compositions should optimally retain the activities of IL-10 beneficial to treatment of the listed disorders but not other, less desirable activities.

### SUMMARY OF THE INVENTION

IL-10 peptides, like IL-10 protein, appear to selectively inhibit products of glial activation that lead to pathology while leaving basal glial and neuronal functions unaltered. Thus, the delivery of IL-10 peptides to the central nervous system may be employed as part of a general therapy for treating neuroinflammation and related disorders.

In one embodiment, the invention relates to methods of treating a pathological condition in a vertebrate subject by administering to the CNS of the subject a peptide containing a sequence derived from an anti-inflammatory cytokine, a cytokine antagonist or an agent that acts to prevent pro-inflammatory cytokine actions. In some embodiments the anti-inflammatory cytokine is one or more cytokines selected from the group consisting of interleukin-10 (IL-10), interleukin-1 receptor antagonist (IL-1ra), interleukin-4 (IL-4), interleukin-2 (IL-2), interleukin-13 (IL-13), tumor necrosis factor soluble receptor (TNFsr), alpha-MSH, and transforming growth factor-beta 1 (TGF-β1). In one embodiment the cytokine is IL-10.

In one embodiment of the present invention, the IL-10 peptide is administered to the central nervous system, e.g. via intrathecal, intraparenchymal, intracerebroventricular, intranasal, or other mechanisms, to human subjects for the treatment of a pathological condition in the subject. In one embodiment, the pathological condition is a neuropathic pain syndrome. In one embodiment, the pain syndrome is trigeminal neuralgia (TGN). In another embodiment, the pathological condition is a chronic neurodegenerative disease. In exemplary embodiments, the chronic neurodegenerative disease is selected from among the group consisting of Alzheimer's disease (AD), Parkinson's disease (PD), amyotrophic lateral sclerosis (ALS) and multiple sclerosis (MS).

In one embodiment, the IL-10-derived peptide comprises the sequence Ala-Tyr-Met-Thr-Met-Lys-Ile-Arg-Asn (written in the conventional amino-to-carboxy terminal format) (SEQ ID NO: 4) or the sequence X1-X2-X3-Thr-X4-Lys-X5-Arg-X6 (SEQ ID NO: 5) where X1=Ala or Gly; X2=Tyr or Phe; X3, X4 and X5 are independently selected from Met, Ile, Leu and Val; and X6-Asp, Gln or Gly.

In another embodiment, IL-10-derived peptides of the present invention are modified to extend their CNS half-life and duration of action, e.g. via PEGylation.

In still further embodiments, bioactive IL-10-derived peptides are used in combination therapy with one or more other agents for treatment of pain and/or chronic neurodegenerative conditions. In a preferred embodiment, the other agent is selected from among those agents having a different mechanism of action in treatment of those specified conditions. In one embodiment, the other agent is selected from the group consisting of carabmazepine (Tegretol®), phenytoin (Dilantin®), oxycarbazepine (Trileptal®), baclofen (Lioresal®) and gabapentin (Neurotonin®).

### BRIEF DESCRIPTION OF THE FIGURES

FIG. 1 shows a comparison of the amino acid sequences of mature secreted forms of human IL-10 (hIL-10) (SEQ ID NO: 1), mouse IL-10 (mIL-10) (SEQ ID NO:2) and a viral form of IL-10 (vIL-10) (SEQ ID NO:3). Amino acid residues differing from the human sequence are boxed.

### DETAILED DESCRIPTION

### I. Definitions

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “an anti-inflammatory cytokine” includes a mixture of two or more such cytokines, and the like.

The term “nervous system” includes both the central nervous system and the peripheral nervous system.” The term “central nervous system” or “CNS” includes all cells and tissue of the brain and spinal cord of a vertebrate. The term “peripheral nervous system” refers to all cells and tissue of the portion of the nervous system outside the brain and spinal cord. Thus, the term “nervous system” includes, but is not limited to, neuronal cells, glial cells, astrocytes, cells in the cerebrospinal fluid (CSF), cells in the interstitial spaces, cells in the protective coverings of the spinal cord, epidural cells (i.e., cells outside of the dura mater), cells in non-neural tissues adjacent to or in contact with or innervated by neural tissue, cells in the epineurium, perineurium, endoneurium, funiculi, fasciculi, and the like.

The term “anti-inflammatory cytokine” as used herein refers to a protein that decreases the action or production of one or more proinflammatory cytokines or proteins produced by nerves, neurons, glial cells, endothelial cells, fibroblasts, muscle, immune cells or other cell types. Such inflammatory cytokines and proteins include, without limitation, interleukin-1 beta (IL-1β), tumor necrosis factor-alpha (TNF-α), interleukin-6 (IL-6), inducible nitric oxide synthetase (iNOS) and the like. Non-limiting examples of anti-inflammatory cytokines include interleukin-10 (IL-10) including viral IL-10, interleukin-4 (IL-4), interleukin-13 (IL-13), alpha-MSH, transforming growth factor-beta 1 (TGF-β1), and the like. Active fragments derived from any of these anti-inflammatory cytokines, as well as active analogs thereof, which retain the ability to decrease pain, such as to treat trigeminal neuralgia, as measured in any of the known pain models including those described further herein, are intended for use with the present invention.

Thus, fragments of these proteins, as well as fragments with modifications, such as deletions, additions and substitutions (either conservative or non-conservative in nature), to the native sequence, are intended for use herein, so long as the peptide maintains the desired activity. These modifications may be deliberate, as through site-directed mutagenesis, or may be accidental, such as through mutations of hosts which produce the proteins or errors due to PCR amplification. Accordingly, active peptides substantially homologous to the parent sequence, e.g., peptides with 70 . . . 80 . . . 85 . . . 90 . . . 95 . . . 98 . . . 99% etc. identity that retain the ability to reduce pain, are contemplated for use herein.

By “proinflammatory cytokine antagonist” is meant any molecule that blocks or antagonizes the biologic action of a proinflammatory cytokine, such as by binding or interacting with a proinflammatory cytokine receptor thereby reducing or inhibiting the production of the proinflammatory cytokine. The terms “antagonist”, “inhibitor”, and “blocker” are used interchangeably herein. Non-limiting examples of such antagonists include interleukin-1 receptor antagonist (IL-1ra); KINERET (recombinant IL-1ra, Amgen); tumor necrosis factor soluble receptor (TNFsr); soluble TNF receptor Type I (Amgen); pegylated soluble TNF receptor Type I (PEGs TNF-R1) (Amgen); TNF decoy receptors; ETANERCEPT (ENBREL, Amgen); INFLIXIMAB (REMICADE, Johnson & Johnson); D2E7, a human anti-TNF monoclonal antibody (Knoll Pharmaceuticals, Abbott Laboratories); CDP 571 (a humanized anti-TNF IgG4 antibody); CDP 870 (an anti-TNF alpha humanized monoclonal antibody fragment), both from Celltech; ONERCEPT, a recombinant TNF binding protein (r-TBP-1) (Serono); IL1-Receptor Type 2 (Amgen), AMG719 (Amgen) and IL-1 Trap (Regeneron).

Active fragments of any of these proinflammatory cytokine antagonists, as well as active analogs thereof, which retain the ability to decrease pain, such as to treat trigeminal neuralgia as measured in any of the known pain models including those described further herein, are intended for use with the present invention.

Thus, fragments of these proteins, as well as fragments with modifications, such as deletions, additions and substitutions (either conservative or non-conservative in nature), to the native sequence, are intended for use herein, so long as the peptide maintains the desired activity. These modifications may be deliberate, as through site-directed mutagenesis, or may be accidental, such as through mutations of hosts which produce the proteins or errors due to PCR amplification. Accordingly, active peptides substantially homologous to the parent sequence, e.g., peptides with 70 . . . 80 . . . 85 . . . 90 . . . 95 . . . 98 . . . 99% etc. identity that retain the ability to reduce pain, are contemplated for use herein.

By “an agent that acts to reduce inflammatory cytokine actions” is meant an agent that induces anti-inflammatory cytokine production. Such agents include, without limitation, IL-9, Hsp27 (see, U.S. Patent Publication No. 2001/0049357).

Thus, fragments of these proteins, as well as fragments with modifications, such as deletions, additions and substitutions (either conservative or non-conservative in nature), to the native sequence, are intended for use herein, so long as the peptide maintains the desired activity. These modifications may be deliberate, as through site-directed mutagenesis, or may be accidental, such as through mutations of hosts which produce the proteins or errors due to PCR amplification. Accordingly, active peptides substantially homologous to the parent sequence, e.g., peptides with 70 . . . 80 . . . 85 . . . 90 . . . 95 . . . 98 . . . 99% etc. identity that retain the ability to reduce pain, are contemplated for use herein.

The term “analog” refers to biologically active derivatives of the reference molecule, or fragments of such derivatives, that retain the ability to reduce pain. In general, the term “analog” refers to compounds having a native polypeptide sequence and structure with one or more amino acid additions, substitutions and/or deletions, relative to the native molecule. Particularly preferred analogs include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 or 50 conservative or non-conservative amino acid substitutions, or any number between 5-50, so long as the desired function of the molecule remains intact.

By “peptide” is meant a fragment of a reference protein that does not include the entire sequence of the protein. Thus, a peptide can be as short as 5 amino acids in length, such as 5 to 150 amino acids in length, such as 7 to 100 amino acids in length, more particularly 5, 6, 7, 8, 9, 10 . . . 20 . . . 30 . . . 40 . . . 50 . . . 60 . . . 70 . . . 80 . . . 90 . . . 100 . . . 150, or any integer within these ranges.

“Homology” refers to the percent identity between two polynucleotide or two polypeptide moieties. Two DNA, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified DNA or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National Biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are well known in the art.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

A “therapeutically effective amount” means an amount of a compound that, when administered to a subject for treating a neurodegenerative disorder or for treating pain, such as trigeminal neuralgia, is sufficient to effect such treatment for the disease. The “therapeutically effective amount” will vary depending on the compound, and disorder being treated, the severity of the disorder treated, the age and relative health of the subject, the route and form of administration, the judgment of the attending medical or veterinary practitioner, and other factors.

The term “pharmacological effect” as used herein encompasses effects produced in the subject that achieve the intended purpose of a therapy. In one preferred embodiment, a pharmacological effect means that pain symptoms of the subject being treated are prevented, alleviated, or reduced. For example, a pharmacological effect would be one that results in the reduction of pain in a treated subject. In another preferred embodiment, a pharmacological effect means that disorders or symptoms of the neurodegenerative disorder of the subject being treated are prevented, alleviated, or reduced.

“Treating” or “treatment” of a disorder includes:

(1) preventing the disorder, i.e. causing the clinical symptoms of the disease not to develop in a subject that may be exposed to or predisposed to the disease, but does not yet experience or display symptoms of the disease,

(2) inhibiting the disorder, i.e., arresting the development of the disorder or its clinical symptoms, or

(3) relieving the disorder, i.e., causing regression of the disorder or its clinical symptoms.

The term “subject” as used herein encompasses mammals and non-mammals. Examples of mammals include, but are not limited to, any member of the Mammalia class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds and the like. The term does not denote a particular age or sex.

### II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention involves administration of peptides derived from cytokines, such as IL-10, which is a potent modulator of microglial responses in brain, in therapy for neurodegenerative diseases such as AD, PD, MS and ALS, each of which diseases involves an inflammatory response that IL-10 may attenuate. The present invention also relates to administration of the same peptides for treatment of neuropathic pain syndromes, such as TGN. One such IL-10-derived peptide, having the sequence Ala-Tyr-Met-Thr-Met-Lys-Ile-Arg-Asn (SEQ ID NO: 4), is disclosed in U.S. Pat. No. 6,159,937, the disclosure of which is hereby incorporated by reference in its entirety. Other IL-10 peptide sequences, disclosed in WO 97/26778 (also incorporated by reference in its entirety) have the sequence X1-X2-X3-Thr-X4-Lys-X5-Arg-X6 (SEQ ID NO: 5) where X1=Ala or Gly; X2=Tyr or Phe; X3, X4 and X5 are independently selected from Met, Ile, Leu and Val; and X6=Asp, Gln or Gly.

The family of other neurological disorders that may be treatable by peptides derived from IL-10, or other anti-inflammatory cytokines, includes but is not limited to fatal familial insomnia, Rasmussen's encephalitis, Down's syndrome, Huntington's disease, Gerstmann-Straussler-Scheinker disease, tuberous sclerosis, neuronal ceroid lipofuscinosis, subacute sclerosing panencephalitis, Lyme disease; tse tse's disease (African Sleeping Sickness), reflex sympathetic dystrophy, complex regional pain syndrome, HIV dementia, bovine spongiform encephalopathy (“mad cow” disease); Creutzfeldt Jacob disease; Herpes simplex encephalitis, Herpes Zoster cerebellitis, general paresis (syphilis), tuberculous meningitis, tuberculous encephalitis, optic neuritis, granulomatous angiitis, temporal arthritis, cerebral vasculitis, Spatz-Lindenberg's disease, methamphetamine-associated vasculitis, cocaine-associated vasculitis, traumatic brain injury, stroke, Lance-Adams syndrome, post-anoxic encephalopathy, radiation necrosis, limbic encephalitis, progressive supranuclear palsy, striatonigral degeneration, corticocobasal ganglionic degeneration, primary progressive aphasia, frontotemporal dementia associated with chromosome 17, spinal muscular atrophy, HIV-associated myelopathy, HTLV-1-associated myelopathy (Tropical Spastic Paraparesis), tabes dorsalis (syphilis), transverse myelitis, post-polio syndrome, spinal cord injury, radiation myelopathy, Charcot-Marie-Tooth, HIV-associated polyneuropathies, campylobacter-associated motor axonopathies, Guillian Barre Syndrome, chronic inflammatory demyelinating polyneuropathy, diabetic amyotrophy avulsion, phantom limb, complex regional pain syndrome, diabetic neuropathies, paraneoplastic neuropathies, myotonic dystrophy, HTLV-1-associated myopathy, trichinosis, inflammatory myopathies (polymyositis, inclusion body myositis, dermatomyositis), sickle cell disease, alpha-1-antitrypsin deficiency, tuberculosis, subacute bacterial endocarditis, chronic viral hepatitis, viral cardiomyopathy, Chaga's disease, malaria, Coxsackie B infection, macular degeneration, retinitis pigmentosa, and vasculitis.

The IL-10-derived peptides of the present invention can be modified to improve their pharmacologic properties using methods known in the art. The most common modification employed to increase the effective half-life of proteins, thereby increasing their utility, is their conjugation with polyethylene glycol (PEG). PEGylation techniques are well known in the art and include, for example, site-specific pegylation (see, e.g., Yamamoto et al. (2003) Nat. Biotech. 21:546-552; Manjula et al. (2003) Bioconjug. Chem. 14:464-72; Goodson and Katre (1990) Biotechnology 8:343-46; U.S. Pat. No. 6,310,180), pegylation using size exclusion reaction chromatography (see, e.g., Fee, C. J. (2003) Biotechnol. Bioeng. 82:200-06), and pegylation using solid phase (see, e.g., Lu and Felix (1993) Pept. Res. 6:140-146). For other methods of pegylation see, e.g., International Publication No. WO 02/26265, U.S. Pat. Nos. 5,206,344 and 6,423,685, as well as reviews by Harris and Chess (2003) Nat. Rev. Drug. Discov. 2:214-21; Greenwald et al. (2003) Adv. Drug. Deliv. Rev. 55:217-56; and Delgado et al. (1992) Crit. Rev. Ther. Drug Carrier Syst. 9:249-304.

PEGylation decreases the clearance of the conjugated polypeptide, resulting in a more sustained effective plasma concentration and thus increased bioavailability of the polypeptide (Eliason, J. F. (2001)Biodrugs 15:705-711; Francis, G. E., et al. (1998) Intl. J. Hematol. 68:1-18; Harris, J. M. (2001) Clin. Pharmcokinet. 40:539-551; WO 02/26265 A2). PEGylation has been demonstrated to alter favorably the pharmacokinetic profiles of proteins, and may also result in more localized absorption of the conjugate. An additional benefit may be reduced immunogenicity of the conjugated protein due to a steric reduction of the access of the immune recognition system to the conjugated polypeptide.

Other cytokines or regulatory proteins can also serve as the source of peptide sequences for use in the methods of the present invention. Such proteins include, for example, interleukin-1 receptor antagonist (IL-1ra), interleukin-4 (IL-4), interleukin-2 (IL-2), interleukin-13 (IL-13), tumor necrosis factor soluble receptor (TNFsr), alpha-MSH, and transforming growth factor-beta 1 (TGF-β1). For example, the C-terminal 13-mer of IL-2 (Trp-Ile-Thr-Phe-Cys-Gln-Ser-Ile-Ile-Ser-Thr-Leu-Thr) (SEQ ID NO:6) has been shown to be essential for at least some IL-2 activities (Ju et al. (1987) J. Biol. Chem. 262(12):5723-31), and thus may also be useful in treatment of neuropathic pain, including TGN, and neurodegenerative disorders.

Small molecule compounds with activities or methods of action similar to IL-10 peptide may also be useful for the treatment of neuropathic pain and/or chronic neurodegenerative conditions. For example, thalidomide reduces thermal hyperalgesia and mechanical allodynia in the chronic constriction injury model of neuropathic pain. George et al. (2000) Pain 88(3):267-75. Minocycline is a selective microglial inhibitor that is effective in blocking induction of neuropathic pain, but not in reversing established neuropathic pain. See Milligan et al. (2005) Molecular Pain 1:9.

The invention also relates to bioactive IL-10-derived peptides used in combination therapy with one or more other agents for treatment of pain and/or chronic neurodegenerative conditions. Such other agents include, but are not limited to, carabmazepine (Tegretol®), phenytoin (Dilantin®), oxycarbazepine (Trileptal®), baculofen (Lioresal®) and gabapentin (Neurotonin®). These other agents are of particular use in the treatment of TGN. Further examples of pain relievers that can be used with the present methods are detailed below. Preferably the other agent has a different mechanism of action in treatment of the specified condition than the IL-10 derived peptide.

The other agents may be used in therapy at doses ranging from 200 to over 1600 mg per patient per day, for example divided into twice-daily doses. For example, an IL-10 peptide can be administered to a human subject in combination with carbamazepine, wherein the carbamazepine is administered in 100 or 200 mg tablets three or four times a day.

In order to further an understanding of the invention, a more detailed discussion is provided below regarding anti-inflammatory cytokines, as well as various compositions and delivery methods for use with the present invention.

Anti-inflammatory Cytokines, Proinflammatory Cytokine Antagonists and Agents that act to Reduce or Prevent Inflammatory Cytokine Action

As explained above, the present invention makes use of peptides derived from anti-inflammatory cytokines, proinflammatory cytokine antagonists and agents that act to reduce or prevent inflammatory cytokine action, to treat TGN and neurodegenerative disorders. Particularly preferred anti-inflammatory cytokines and antagonists for deriving peptides for use with the present invention include, without limitation, interleukin-10 (IL-10), interleukin-1 receptor antagonist (IL-1ra), interleukin-4 (IL-4), interleukin-13 (IL-13), tumor necrosis factor soluble receptor (TNFsr), alpha-MSH and transforming growth factor-beta 1 (TGF-β1). Fragments of the native molecules, as well analogs thereof, which retain the desired biological activity, are intended for use with the present invention. Moreover, sequences derived from any of numerous species can be used with the present invention, depending on the animal to be treated.

For example, fragments from a number of sequences related to IL-10, as well as fragments from variants and agonists, with the desired function will also find use herein. For example, sequences related to IL-10 are described in, e.g., International Publication Nos. WO 00/65027; WO 98/28425; WO 95/24425 (immunomodulator Trichinella substances). International Publication No. WO 95/03411 describes shortened IL-10 sequences, variants and agonists of IL-10 having amino acid substitutions or deletions at the carboxyl and/or amino terminus of mature human sequence; U.S. Pat. No. 6,428,985 describes IL-10 variants with a substitution of Ile at position 87 of the mature human IL-10 sequence with Ala or Gly; U.S. Pat. No. 6,159,937 describes an IL-10 fragment with the sequence Ala-Tyr-Met-Thr-Met-Lys-Ile-Arg-Asn) (SEQ ID NO:4); International Publication No. WO 97/26778 describes IL-10 variants with the sequence X1-X2-X3-Thr-X4-Lys-X5-Arg-X6 (SEQ ID NO:5) where X1=Ala or Gly; X2=Tyr or Phe; X3, X4 and X5 are independently selected from Met, Ile, Leu and Val; and X6=Asp, Gln or Gly.

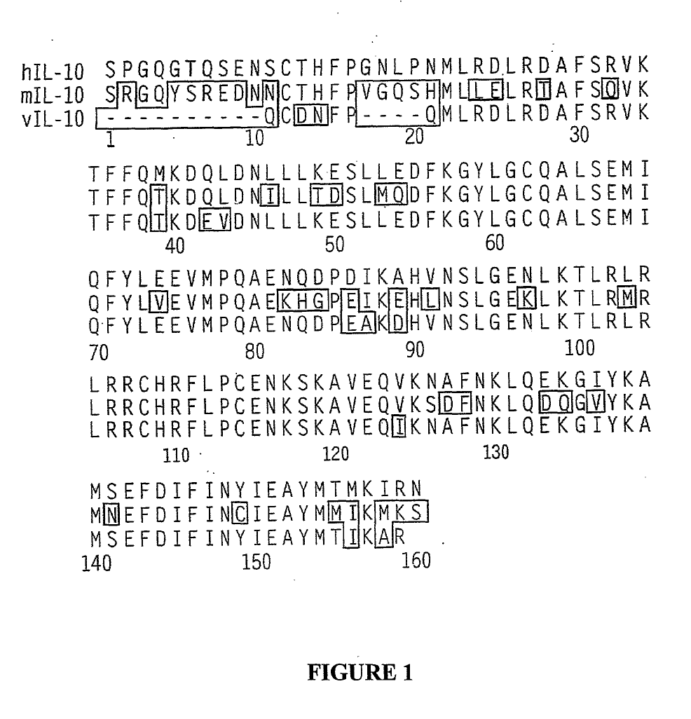

Nucleotide and amino acid sequences of anti-inflammatory cytokines, proinflammatory cytokine antagonists and agents that act to reduce or prevent inflammatory cytokine action, and variants thereof, from several animal species are well known. For example, IL-10 has been isolated from a number of animal and viral species. Fragments of IL-10 for use herein include IL-10 fragments from any of these various species. Non-limiting examples of viral IL-10 include the IL-10 homologues isolated from the herpesviruses such as from Epstein-Barr virus (see, e.g., Moore et al., Science (1990) 248:1230-1234; Hsu et al., Science (1990) 250:830-832; Suzuki et al., J. Exp. Med. (1995) 182:477-486), Cytomegalovirus (see, e.g., Lockridge et al., Virol. (2000) 268:272-280; Kotenko et al., Proc. Natl. Acad. Sci. USA (2000) 97:1695-1700; International Publication No. WO 01/16153), and equine herpesvirus (see, e.g., Rode et al., Virus Genes (1993) 7:111-116), as well as the IL-10 homologue from the OrF virus (see, e.g., Imlach et al., J. Gen. Virol. (2002) 83:1049-1058 and Fleming et al., Virus Genes (2000) 21:85-95). See, also, FIG. 1 herein depicting the amino acid sequence of a mature, secreted form of viral IL-10. Representative, non-limiting examples of other IL-10 sequences for deriving peptides for use with the present invention include the sequences described in NCBI accession numbers NM000572, U63015, AF418271, AF247603, AF247604, AF247606, AF247605, AY029171, UL16720 (all human sequences), and FIG. 1 herein depicting the amino acid sequence of a mature secreted form of human IL-10; NM012854, L02926, X60675 (rat); NM010548, AF307012, M37897, M84340 (all mouse sequences), and FIG. 1 herein depicting the amino acid sequence of a mature secreted form of mouse IL-10; U38200 (equine); U39569, AF060520 (feline sequences); U00799 (bovine); U11421, Z29362 (ovine sequences); L26031, L26029 (macaque sequences); AF294758 (monkey); U33843 (canine); AF088887, AF068058 (rabbit sequences); AF012909, AF120030 (woodchuck sequences); AF026277 (possum); AF097510 (guinea pig); U11767 (deer); L37781 (gerbil); AB107649 (llama and camel).

Non-limiting examples of IL-1ra sequences for deriving peptides for use with the present invention include the sequences described in NCBI accession numbers NM173843, NM173842, NM173841, NM000577, AY196903, BC009745, AJ005835, X64532, M63099, X77090, X52015, M55646 (all human sequences); NM174357, AB005148 (bovine sequences); NM031167, S64082, M57525, M644044 (mouse sequences); D21832, 568977, M57526 (rabbit sequences); SEG AB045625S, M63101 (rat sequences); AF216526, AY026462 (canine sequences); U92482, D83714 (equine sequences); AB038268 (dolphin).

Non-limiting examples of IL-4 sequences for deriving peptides for use with the present invention include the sequences described in NCBI accession numbers NM172348, AF395008, AB015021, X16710, A00076, M13982, NM000589 (all human sequences); BC027514, NM021283, AF352783, M25892 (mouse sequences); NM173921, AH003241, M84745, M77120 (bovine sequences); AY130260 (chimp); AF097321, L26027 (monkey); AY096800, AF172168, Z11897, M96845 (ovine sequences); AF035404, AF305617 (equine sequences); AF239917, AF187322, AF054833, AF104245 (canine sequences); X16058 (rat); AF046213 (hamster); L07081 (cervine); U39634, X87408 (feline); X68330, L12991 (porcine sequences); U34273 (goat); AB020732 (dolphin); L37779 (gerbil); AF068058, AF169169 (rabbit sequences); AB107648 (llama and camel).

Non-limiting examples of IL-13 sequences for deriving peptides for use with the present invention include the sequences described in NCBI accession numbers NM002188, U10307, AF377331, X69079 (all human sequences); NM053828, L26913 (rat sequences); AF385626, AF385625 (porcine sequences); AF244915 (canine); NM174089 (bovine); AY244790 (monkey); NM008355 (mouse); AB107658 (camel); AB107650 (llama).

Non-limiting examples of TGF-β1 sequences for deriving peptides for use with the present invention include the sequences described in NCBI accession numbers NM000660, BD0097505, BD0097504, BD0097503, BD0097502 (all human sequences); NM021578, X52498 (rat sequences); AJ009862, NM011577, BC013738, M57902 (mouse sequences); AF461808, X12373, M23703 (porcine sequences); AF175709, X99438 (equine sequences); X76916 (ovine); X60296 (hamster); L34956 (canine).

Non-limiting examples of alpha-MSH sequences for deriving peptides for use with the present invention include the sequences described in NCBI accession number NM 000939 (human); NM17451 (bovine); NM 008895 (mouse); and M 11346 (xenopus).

Non-limiting examples of TNF receptor sequences for deriving peptides for use with the present invention include the sequences described in NCBI accession numbers X55313, M60275, M63121, NM152942, NM001242, NM152877, NM152876, NM152875, NM152874, NM152873, NM152872, NM152871, NM000043, NM 001065, NM001066, NM148974, NM148973, NM148972, NM148971, NM148970, NM148969, NM148968, NM148967, NM148966, NM148965, NM003790, NM032945, NM003823, NM001243, NM152854, NM001250 (all human sequences); NM013091, M651122 (rat sequences).

Non-limiting examples of IL-9 sequences for deriving peptides for use with the present invention include the sequences described in NCBI accession numbers NM000590 (human) and NM008373 (mouse).

Compositions and Delivery

A. Compositions

Compositions will comprise a therapeutically effective amount of the anti-inflammatory cytokine of interest, i.e., an amount sufficient to reduce or ameliorate pain such as caused by TGN or to treat the neurodegenerative disorder of interest. The compositions will also contain a pharmaceutically acceptable excipient. Such excipients include any pharmaceutical agent that does not itself harm the individual receiving the composition, and which may be administered without undue toxicity. Pharmaceutically acceptable excipients include, but are not limited to, sorbitol, any of the various TWEEN compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in REMINGTON'S PHARMACEUTICAL SCIENCES (Mack Pub. Co., N.J. 1991).

As is apparent to those skilled in the art in view of the teachings of this specification, an effective amount can be empirically determined. Administration can be effected in one dose, continuously or intermittently throughout the course of treatment. Methods of determining the most effective means and dosages of administration are well known to those of skill in the art and will vary with the composition of the therapy, the target cells, and the subject being treated. Single and multiple administrations can be carried out with the dose level and pattern being selected by the treating physician.

If multiple doses are administered, the first formulation administered can be the same or different than the subsequent formulations. Moreover, subsequent delivery can also be the same or different than the second mode of delivery.

Furthermore, as explained above, the methods of the present invention can be combined with other suitable compositions and therapies. Such other agents include, but are not limited to, carabmazepine (Tegretol®), phenytoin (Dilantin®), oxycarbazepine (Trileptal®), baculofen (Lioresal®) and gabapentin (Neurotonin®). Additional pain alleviators and analgesics, such as anti-prostaglandins, including, without limitation, cyclooxygenase-2 (COX-2) inhibitors, 5-lipoxygenase (5-LOX) inhibitors, and the like, can be coadministered with the compositions of the invention. Other compounds for delivery include agents used in the treatment of neuropathic pain such as, but not limited to, tricyclic antidepressants (e.g., amitriptyline, imipramine, desipramine), anti-convulsants (e.g., gabapentin, carbamazepine, phenytoin) and local anesthetics (e.g., mexiletine, lidocaine); and agents used in the treatment of inflammatory pain including, but not limited to, NSAIDs (e.g., ibuprofen, naprosyn sodium, aspirin, diclofenac sodium, indomethacin, toletin), steroids (e.g., methylprednisone, prednisone), analgesics (e.g., acetaminophen), and opiates (e.g., tramadol, demerol, darvon, vicodin, fentanyl).

B. Delivery

The mode of delivery of the compositions of the present invention can be any mode capable of delivering to the CNS. Intrathecal delivery overcomes the blood-brain barrier (BBB) by direct injection into the cerebrospinal fluid. The peptide is released into the surrounding CSF and/or tissues and can penetrate into the spinal cord parenchyma, just as after acute intrathecal injections.

Intranasal delivery (IND) is a noninvasive alternative method of bypassing the BBB to deliver therapeutic agents to the brain and spinal cord, eliminating the need for systemic delivery and thereby reducing unwanted systemic side effects. IND works because of the unique connection between the nerves involved in sensing odors and the external environment. Delivery from the nose to the central nervous system takes place within minutes along both the olfactory and trigeminal neural pathways. Delivery occurs by an extracellular route and does not require that the drugs bind to any receptor or undergo axonal transport. Bulk flow through perivascular and hemangiolymphatic channels may also be involved in the movement of drugs from the nose to the brain and spinal cord. The precise mechanism of IND is not an important element of the invention. IND is of particular interest in the treatment of orofacial pain variously diagnosed as idiopathic and secondary trigeminal neuralgia, peripheral neuropathy and reflex sympathetic dystrophy. Trauma, dental treatments, orofacial surgery and MS have also been linked to orofacial chronic neurpathic pain syndromes.

IND may be effected, for example, using nasal sprays, drops or powders. Devices for IND include nasal sprays, nose drips, saturated cotton pledgets, aerosol sprays and insufflators. Nebulizers, such as the Ultravent™ (Mallickrodt Inc., St. Louis, Mo.) may be used. Viscosity enhancing agents such as methylcellulose can be added to a formulation of peptide to change the pattern of deposition and clearance during IND. Bioadhesives can also be added to increase the residence time in the nasal cavity. Various formulations and devices for IND are described at Chein (1987) Crit. Rev. Therap. Drug Carr. Sys. 4:67 and Wearley (1991) Crit. Rev. Therap. Drug Carr. Sys. 8:331-94.

Another method for delivery is by administration into the epidural space. The epidural space occupies the vertebral canal between the periosteum lining the canal and the dura. The epidural space is readily approached through the lumbar area. Generally, a needle, catheter or the like is inserted in the midline and passes through the skin, fascia, supraspinous and interspinous ligaments, and the ligamentum flavum prior to reaching the extradural space. However, administration can also be through the thoracic area. Methods for delivering agents epidurally are well known in the art. See, e.g., Textbook of Surgery, D. C. Sabiston, ed.) W.B. Saunders Company.

Another preferred method for administering the compositions is by delivery to dorsal root ganglia (DRG) neurons, e.g., by injection into the epidural space with subsequent diffusion to DRG. For example, the compositions can be delivered via intrathecal cannulation under conditions where the protein is diffused to DRG. See, e.g., Chiang et al., Acta Anaesthesiol. Sin. (2000) 38:31-36; Jain, K. K., Expert Opin. Investig. Drugs (2000) 9:2403-2410.

### III. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLE 1

### Oligopeptide Manufacture

Oligopeptide manufacture is achieved by solid-phase synthesis methods known to those skilled in the art. Analysis of the synthesized oligopeptides includes electrospray mass spectrometry, high performance liquid chromatography, and visual appearance of the purified product. The oligopeptide(s) are prepared in water for injection at 1 mg/ml. An example of a proper IL-10-derived peptide (see U.S. Pat. No. 6,159,937) and a “scrambled” control peptide are provided in Table 1. Peptide sequences are provided in the conventional N→C terminal direction. Amino acids are named using the three-letter nomenclature.

TABLE 1

| | |
|---|---|
| Human IL-10 peptide (IT) | Ala-Tyr-Met-Thr-Met- |
| (SEQ ID NO: 4) | Lys-Ile-Arg-Asn |
| ‘Scrambled’ peptide (S) | Arg-Ile-Lys-Asn-Met- |
| (SEQ ID NO: 7) | Ala-Thr-Tyr-Met |

Although an exemplary peptide sequence is provided in Table 1, it would be clear to one of skill in the art that various modifications or substitutions could be made to the listed sequence which would retain, and perhaps improve, the efficacy of the peptide in treatment of neuropathic pain or neurodegenerative disease, or improve its pharmacologic properties. Such sequence variants could be tested in one or more of the models described in the following examples to assess their therapeutic efficacy.

For example, IL-10 sequences from non-human species could be used to obtain IL-10-derived peptide sequences differing from the human-derived IL-10 peptide, which non-human IL-10-derived peptides may exhibit improved properties compared to the human-derived sequence. Alternatively, variants can be designed by inspection using known empirical parameters familiar to those of skill in the art of therapeutic peptides. Additionally, rational drug design can be used to design a sequence variant that would be expected to exhibit increased efficacy, which rational drug design can be based on analysis of the three dimensional structure of an IL-10, an IL-10 receptor, or a complex of IL-10 with a receptor.

### EXAMPLE 2

### Efficacy of IL-10 Peptide in the Chung Model of Neuropathic Pain

### Experimental Model:

Rats are individually anesthetized using 5% isoflurane in a plexiglass chamber and are then transferred to a mask and maintained at 2.5-3% isoflurane depending upon repiratory rate. Throughout the procedure, animals are maintained on a heating pad warmed to 37° C. Rats are shaved from above the hips to over the left thigh including the area in-between. The shaved area is swabbed with Betadyne and an incision is made from the hip to knee in a straight line using a number 10 blade being careful not to incise the muscle. The common sciatic nerve is exposed at the level of the middle of the thigh by blunt dissection through biceps femoris. Skin is clamped away from the site and the incision is draped. Glass hooks are used to tease tissue away from nerve and 4 ligatures of 4-0 chromic gut is tied loosely around the nerve such that the suture slides along nerve when knotted but not so loose that space can be seen between suture and nerve. The nerve is dropped back into the muscle trench and the muscle is closed in layers. The skin is closed with two staples. Following surgery, animals are allowed to recover from the anesthetic in a clean, plexiglass box containing a heating pad. When alert, rats are returned to their littermates in cages containing clean shavings. A waiting period of 72 h commences prior to behavioral testing. Overall well-being of the animal is checked immediately following and at every 24 h post-surgery.

### Experimental Design:

Animals are divided into one of eight groups. Groups 1-5 are administered doses of IL-10 peptide (referred to herein as “IT”, comprising the C-terminal nonapeptide from human IL-10 protein) of 10 μg, 1 μg, 0.1 μg, 0.01 μg and 0.001 μg, respectively. (Unless otherwise indicated by the context, “IT” as used herein refers to the IL-10 peptide referenced above, and does not refer to “intrathecal” delivery.) Groups 6-8 are included as controls. Group 6 animals are administered 10 μg of a “scrambled” peptide (referred to as “S”) having the same amino acid composition as peptide IT but in randomized order. Group 7 is administered vehicle alone, and Group 8 is sham-operated and then administered 10 μg of peptide IT.

### Route of Peptide Administration:

Direct delivery: Rats undergo gas anesthesia (2.5% isoflurane carried in 100% oxygen) for the injection procedure. The animal is placed in a prone position with the pelvic girdle slightly elevated. To visualize the area and allow identification of landmarks (L5-L6 spinous processes and the pelvic girdle), a small region of the back is shaved. Using aseptic procedure, a hypodermic needle (30 g×½″) attached to a Hamilton syringe (50 ul gas-tight) is carefully and slowly inserted into the groove between the spinous and transverse L5-L6 processes at an angle of approximately 20% and then advanced at an angle of approximately 10% through the dura (approximately 0.5 cm of the needle tip). A volume of up to 50 ul is injected slowly (injection time <20 minutes) to test the substance P reaction for the rat.

Catheter delivery: An 18 ga ½″ hypodermic needle (hub removed) is inserted between vertebrae L5 & L6, piercing the dura. A PE-10 catheter is threaded through the 18 ga guide needle into the lumbo-sacral CSF space. The PE-10 catheter is attached to a 30 ga ⅜″ hypodermic needle, in turn attached to a tuberculin or Hamilton syringe. A volume of up to 50 μl is injected slowly (injection time <20 minutes to test the substance P reaction for the rat.

### Behavioral Measures:

Male Sprague-Dawley rats (230-250 g) are acclimated for 4 days prior to use. The testing room remains at room temperature. Lights are dimmed to our eye acceptable level to limit distress. Rats are acclimated to the handler and apparatus for at least 1 h per day for 4d prior to acquiring pre-Chung baseline measurements. Animals are not restrained during acclimation or throughout the testing period and have full access to food and water prior to and following testing. Baseline measurements for thermal hyperalgesia and mechanical allodynia take place following acclimation and this is considered day-1. Nociceptive testing is conducted as follows: Thermal hyperalgesia testing is conducted after mechanical allodynia testing on the same day between 8 AM and 1 PM to control for variations in animal activity.

Thermal hyperalgesia testing is conducted using a Hargreaves apparatus. Briefly, animals are allowed to acclimate for a minimum of 10 min under a plexiglass enclosure on the surface of a glass plate prior to testing. The toes of the paw ipsilateral to the Chung are heated using a directed light source generated by a lamp set to an infrared intensity such that the baseline (pre-Chung) paw withdrawal latency is 12-15 s. The paw withdrawal latency is defined as the time it takes for the rat to remove its paw from the heat source. The heat source on the Hargreaves apparatus is automatically shut off once the paw is removed. Should the animal not remove the paw, a cutoff threshold of 20 s is implemented to prevent tissue damage. A minimum of two and maximum of three readings separated by 10 min are taken for each rat. Only the ipsilateral paw is tested.

Mechanical allodynia testing is conducted using Von Frey fibers to establish allodynia and an automated Von Frey apparatus to establish threshold in that order. Three potential testing paradigms may be employed. First, rats start on the plantar surface of the ipsilateral paw 10× each without significant time between intervals using the 2 g filament followed 10 min later by 10× each using the 10 g filament. This procedure is repeated three times. The number of positive responses out of 30 is recorded for each filament. A positive response is one in which the rat lifts its paw upon bending of the filament. Second, animals are stimulated 3× separated by at least 5 min on the ipsilateral paw using a mechanical Von Frey device. When the paw is withdrawn, the paw withdrawal threshold is automatically recorded. Third, the plantar surface of the paw is stimulated with a range of Von Frey filaments (e.g., 0.5 g to 15 g bending force) spanning the predicted response range, with the total number of trials being no greater than 18. The rats' responses are tabulated and used to determine the 50% paw withdrawal threshold response for each animal.

A dose response is obtained by comparing results obtained at doses varying from 10 μg, 1 μg, 0.1 μg, 0.01 μg and 0.001 μg of the IL-10 peptide (IT), and comparing the results to those obtained using the scrambled peptide control (S) at a dose of 10 μg and a vehicle-only control. The dose response is useful to both determine the minimum effective dose and in confirming that any observed effect is a result of the IL-10 peptide rather than some other experimental parameter.

### EXAMPLE 3

### Efficacy of IL-10 Peptide in the CCI Model of Neuropathic Pain

Sprague-Dawley rats (300 g males, n=4-6 per group) are prepared by chronic constriction injury (CCI) surgery. Bennett G J and Xie Y K (1988) Pain 33, 87-107. IL-10 peptide IT and scrambled peptide S are solubilized in water and injected at the indicated doses (1 μg in 10-20 μl final volume) intrathecally under brief isoflurane anesthesia (Milligan et al., 2001, J. Neurosci. 21:2808-19). Behavioral measurements for mechanical allodynia (von Frey testing) are carried out throughout the pre- and post-surgical periods. The peptide has been assayed previously for a variety of immunomodulatory functions known for human IL-10 (Osman, mm et al J Egypt Soc Parasitol. 1999; 29(1):13-20), and results show that this peptide shares some, but not all, of the functional activities of intact IL-10, at generally different dosage levels.

### EXAMPLE 4

### Efficacy of IL-10 Peptide in the Modified Chung Model of Neuropathic Pain

### Experimental Model:

Rats are anesthetized by inhalation anesthesia using an induction concentration of 5% isoflurane in 100% O₂. Isoflurane concentration is maintained at approx 2.5% throughout the procedure. To expose the L5 spinal nerve, a 3 cm skin incision is made and the muscle tissue is separated and retracted from the left superior articular and transverse processes. The transverse process is then partially removed. The L5 spinal nerve is gently separated from the L4 spinal nerve using 6-0 silk suture and transected with removal of a 3 mm segment of nerve (to prevent reconnection). The wound is closed using 3-0 polyester suture for the fascia and surgical staples for the skin. Sham surgery is identical to the L5 spinal nerve surgery; however, the L5 spinal nerve is not transected or manipulated in any way following the partial laminectomy.

### Experimental Design, Route of Peptide Administration, Behavioral Measure:

Experimental design, route of peptide administration and behavioral measures are as described in Example 2.

### Endpoint:

Resolution of allodynia is assessed as the experimental endpoint.

### EXAMPLE 5

### Efficacy of IL-10-Derived Peptides in Parkinson's Disease Model

Rotational behavior is analyzed in unilaterally 6-hydroxydopamine (6-OHDA) lesioned rats both prior to and following either intraparenchymal or intracerebroventricular stereotaxic delivery of IL-10-derived peptides. The 6-OHDA rat model has long been considered an appropriate model for studying Parkinson's disease. Acute challenge with dopamine-replacing drugs (such as L-dopa) or dopamine antagonists (such as apomorphine) elicits a rotational response in 6-OHDA-lesioned rats. This rotation is contraversive to the lesion and is considered an anti-parkinsonian effect.

Unilaterally lesioned rats to be used in the experiment are tested for rotational behavior prior to treatment with IL-10-derived peptides. A behavioral minimum of 160 rotations in 30 minutes is required for rats to be used in the experiment. All test rats must also exhibit robust contralateral rotation in response to apomorphine, and intramuscular administration of methyl-DOPA/benseroside (L-dopa) (5 mg/kg) must not induce rotational behavior.

Four such unilaterally lesioned rats are treated with IL-10 peptide IT and another four are treated with a control scrambled peptide S, as described in Table 1. Rotational behavior of both groups of rats in response to L-dopa administration is assessed at various time points after delivery of peptides.

These and other embodiments of the subject invention will readily occur to those of skill in the art in view of the disclosure herein. Although preferred embodiments of the subject invention have been described in some detail, it is understood that obvious variations can be made without departing from the spirit and the scope of the invention as defined herein.

All publications, patents and patent applications cited herein are hereby incorporated by reference in their entireties.

1. A method of treating trigeminal neuralgia in a vertebrate subject comprising administering to the CNS of said subject a biologically active peptide from an anti-inflammatory cytokine, a cytokine antagonist, or an agent that acts to prevent proinflammatory cytokine actions.

2. The method of claim 1, wherein the subject is administered a peptide from an anti-inflammatory cytokine.

3. The method of claim 2, wherein said anti-inflammatory cytokine is one or more cytokines selected from the group consisting of interleukin-10 (IL-10), interleukin-1 receptor antagonist (IL-1ra), interleukin-4 (IL-4), interleukin-2 (IL-2), interleukin-13 (IL-13), tumor necrosis factor soluble receptor (TNFsr), alpha-MSH, and transforming growth factor-beta 1 (TGF-β1).

4. The method of claim 3, wherein said anti-inflammatory cytokine is IL-10.

5. The method of claim 4, wherein said vertebrate subject is a human and said anti-inflammatory cytokine is human IL-10.

6. The method of claim 1, wherein the peptide comprises the sequence of SEQ ID NO:4.

7. The method of claim 6, wherein the peptide consists of the sequence of SEQ ID NO:4.

8. The method of claim 1, further comprising administering to said subject a second therapeutic agent for treatment of trigeminal neuralgia.

9. A method of treating a neurodegenerative disorder in a vertebrate subject comprising administering to the CNS of said subject a biologically active peptide comprising the sequence of SEQ ID NO:4 or a biologically active peptide comprising the sequence of SEQ ID NO:5.

10. The method of claim 9, wherein the condition is a chronic neurodegenerative disease.

11. The method of claim 10, wherein said chronic neurodegenerative disease is selected from the group consisting of Alzheimer's disease (AD), Parkinson's disease (PD), amyotrophic lateral sclerosis (ALS) and multiple sclerosis (MS).

12. The method of claim 11 wherein the subject is administered a peptide consisting of the sequence of SEQ ID NO:4.

13. The method of claim 11, further comprising administering to said subject a second therapeutic agent for treatment of the neurodegenerative disorder.

14. The method of claim 1, wherein said administering is by intraparenchymal delivery.

15. The method of any claim 1, wherein said administering is by intrathecal delivery.

16. The method of claim 1, wherein said administering is by intracerebroventricular delivery.

17. The method of claim 1, wherein said administration is by intranasal delivery.

18. The method of claim 1, wherein the peptide is PEGylated.

19. (canceled)

20. (canceled)

21. The method of claim 9, wherein said administering is by intraparenchymal delivery.

22. The method of claim 9, wherein said administering is by intrathecal delivery.

23. The method of claim 9, wherein said administering is by intracerebroventricular delivery.

24. The method of claim 9, wherein said administration is by intranasal delivery.

25. The method of claim 9, wherein the peptide is PEGylated.
